(19) United States
(12) Patent Application Publication
(10) Pub. No.: US 2025/0020619 A1
(43) Pub. Date: Jan. 16, 2025

(54) METHODS AND KITS FOR ELUTING ANALYTES FROM IMMUNOAFFINITY COLUMNS

(71) Applicant: Waters Technologies Corporation, Milford, MA (US)

(72) Inventors: Lingyun Chen, Westborough, MA (US); Jianmin Liu, Northborogh, MA (US)

(73) Assignee: Waters Technologies Corporation, Milford, MA (US)

(21) Appl. No.: 18/767,095

(22) Filed: Jul. 9, 2024

Related U.S. Application Data
(60) Provisional application No. 63/525,813, filed on Jul. 10, 2023.

(51) Int. Cl. G01N 30/54; G01N 30/02; G01N 30/30; G01N 33/02

(52) U.S. Cl. CPC G01N 30/54; G01N 33/02; G01N 2030/027; G01N 2030/3061

(57) ABSTRACT

The present technology provides a method of eluting an analyte of interest from an immunoaffinity column. The methods of the present technology provide a simplified method for disassociating a bound analyte from the immunoaffinity column and allows for direct downstream processing of the eluate.

### RELATED APPLICATION

This application claims priority to and the benefit of U.S. Provisional Application No. 63/525,813, filed Jul. 10, 2023, the entire disclosure of which is incorporated herein by reference.

### FIELD OF THE TECHNOLOGY

The present disclosure relates methods and kits for eluting analytes from immunoaffinity columns. In particular, the present disclosure relates to methods and kits which improve compatibility of the eluate with downstream analysis.

### BACKGROUND

Immunoaffinity columns have been used for sample purification and enrichment prior to analytical determination (e.g., prior to HPLC analysis, prior to an immunoassay analysis, prior to LC-MS/MS). Immunoaffinity columns combine the use of liquid chromatography with the specific binding of antibodies or related agents to capture analytes of interest. The resulting method can be used in assays for a particular target or for the purification and concentration of analytes of interest prior to further examination by another technique. Immunoaffinity columns are particularly useful in methods for detecting the presence of harmful substances (e.g., toxins, the analyte of interest) in various types of complex samples (e.g., food or crop samples, patient samples, biological samples).

Analytes of interest are usually eluted from immunoaffinity columns by harsh elution solutions to dissociate the analyte from a stationary phase containing the antibodies (which capture the analyte of interest). Examples of typical elution solutions include strong acidic or basic buffers, high salt solutions with strong ionic strength, organic solvents, denaturing reagents and competing ligands or analogs. In many cases, the resulting eluted sample (i.e., eluate) is diluted or buffer exchanged to be compatible with downstream analysis. That is, additional solutions are added to the eluate to condition it for downstream processing. Dilution and/or buffer exchange processes typically degrade sensitivity and precision of results. For example, dilution can impact the sensitivity of a diagnostic test, such as when a purified sample is analyzed by an immunoassay (e.g., ELISA and lateral flow test) resulting in a false negative (i.e., diluted eluate is too diluted for detection of a positive result). And buffer exchange processes are cumbersome and can introduce impurities and matrix effects through the introduction of additional solutions. These additional solutions and processes affect the recovery and precision of the results.

### SUMMARY

The problems associated with modifying or conditioning of eluate to address adverse effects of an elution solution are mitigated by using an alternative means for disassociation of the analyte from the immunoaffinity column. The present technology provides methods and related kits having improved simplicity and performance as compared to conventional elution methods, which introduce harsh solutions and or require conditioning for compatibility with downstream analytical processes. The present technology provides simplicity as no special elution solution formula is needed for disassociation. Rather, the present technology uses thermal energy (i.e., heat) to disassociate the analyte of interest from the stationary phase. Consequently, the elution solution or wash solution can be selected to be compatible directly with downstream processes (e.g., spectroscopy, HPLC, fluorometry, immunoaffinity assays) without any modification to the eluate (i.e., no dilution, no buffer exchange). Eliminating dilution, not only eliminates a processing step and reduces processing time, but also increases sensitivity of the results. Moreover, elimination of buffer exchange processes eliminates time consuming steps that can introduce both impurities and matrix effects. Thus, precision and recovery can be improved by using methods and related kits which use non-chemical disassociation of the analyte of interest from the stationary phase (i.e., antibody containing material) of the immunoaffinity column.

In one aspect, the present technology is directed a method of eluting a bound analyte from an immunoaffinity column. The method comprises passing a sample solution comprising an analyte of interest through the immunoaffinity column to associate a portion of the analyte of interest to stationary phase material of the immunoaffinity column; heating the immunoaffinity column at a temperature between about 65° C. to about 95° C. for a time period between about 5 minutes and about 30 minutes to disassociate the portion of the analyte of interest from the stationary phase material; and rinsing the immunoaffinity column with a solution to create an eluate including the disassociated portion of the analyte of interest.

The above aspect can include one or more of the following features. In an embodiment, the method can further include injecting the eluate into a downstream processing system without modification of the eluate. Some embodiments feature a phosphate buffered saline or running buffer solution for rinsing the immunoaffinity column. Certain embodiments feature conditioning the immunoaffinity column prior to loading the column with the sample solution. Other embodiments do not include column conditioning. The methods can feature heating the column at a temperature of 90° C. for 12 minutes, 10 minutes, 7 minutes, or 5 minutes. Some embodiments feature heating the column at a temperature of 95° C. to 105° C. for less than 5 minutes (e.g., 3 minutes, 1 minute, 30 seconds). In some embodiments, the eluate is injected into a HPLC column. In certain embodiments the eluate is injected on to a lateral flow device. A reader can be used to evaluate peak height of a test line to peak height of a control line. In some embodiments, the eluate is used in an ELISA test. In some embodiments the eluate is analyzed by a fluorometer. In certain embodiments, the eluate is injected into a LC-MS system. The sample solution used in the methods can include a powered food sample, such as cocoa powder. To form a solution, the powdered food sample is mixed with water, an organic solvent, or both. In some embodiments, the powdered food sample is mixed with a salt solution to extract or form a sample solution. The sample solution can be filtered prior to passing it through the immunoaffinity column. In some embodiments the immunoaffinity column is a mycotoxin immunoaffinity column, such as an ochratoxin immunoaffinity column.

In another aspect, the present technology is directed to a kit for eluting a captured analyte from an immunoaffinity column. The kit comprises an immunoaffinity column, a consumable analytical processing device (e.g., a lateral flow test, a liquid chromatography column) having an inlet for injection of the eluate formed using the method in accordance with the present technology; and a vial of a rinse solution compatible with the consumable analytical processing device. The kits of the present technology can be tailored for the detection of a targeted analyte in a complex sample. For example, the kit can be used for testing the level of ochratoxin in a cocoa powder sample. In some embodiments of this aspect, the consumable analytical processing device of the kit comprises a lateral flow test. In certain embodiments, the consumable analytical processing device of the kit comprises a liquid chromatography column.

### BRIEF DESCRIPTION OF THE DRAWINGS

The technology will be more fully understood from the following detailed description taken in conjunction with the accompanying drawings, in which:

FIG. 1 is an image showing various immunoaffinity chromatography columns.

FIG. 2 provides a flow chart illustrating a conventional method for eluting analytes from an immunoaffinity column.

FIG. 3 provides a flow chart illustrating a method in accordance with the present technology for eluting analytes from an immunoaffinity column.

FIG. 4A is an image illustrating a first step of the method shown in FIG. 3.

FIG. 4B is an image illustrating a second step of the method shown in FIG. 3.

FIG. 4C is an image illustrating a third step of the method shown in FIG. 3

FIG. 5 is a table providing peak height results for test lines and control lines using various elution methods.

### DETAILED DESCRIPTION

The present technology is directed to improved methods for eluting an analyte of interest from an immunoaffinity column. The methods are particularly useful in the purification, enrichment, and analysis of analytes that specifically bind to an affinity ligand (i.e., an antibody or antigen).

The term “analyte” or “analyte of interest”, as used herein, refers to any molecule of interest that is desirable to quantify, measure or detect using the methods described herein.

As used herein, the term “sample” refers to any medium that includes the analyte or analyte of interest (e.g., a mycotoxin) to be quantified using the methods according to the present disclosure. A sample may be selected from an agricultural sample, an environmental sample, or a biological sample. A sample may include, but is not limited to, for example, a food substance (e.g., poultry, fresh meat, milk, yogurt, dairy products, bakery products, beverages, juices, cheeses, vegetables, fruit, fish, etc.), an animal feed, a body of water or sewage (e.g., pond, lake, river, ocean, sewage channels, drinking water, etc.), a clinical specimen (e.g., blood, plasma, serum, sputum, tissue, urine, saliva, sample/fluid from the respiratory tract, etc.), soil, and cosmetic and pharmaceutical products (e.g., lotions, creams, ointments, solutions, medicines, eye and ear drops, etc.).

As used herein, the term “immunoaffinity chromatography” refers to a method for separating target antibodies or antigens from a heterogeneous sample solution. It is a column-based method, meaning that the sample solution is flowed through a column. The column contains a stationary material/support which is functionalized with a capture antibody or antigen also known as an affinity ligand. A target protein (e.g., analyte of interest) is adsorbed onto the stationary phase/support via binding to the capture antibody or antigen and is retained while the rest of the sample solution is washed away. As used herein, the term “affinity ligand” refers to a substance (e.g., a functional group) that selectively captures (binds to) a target molecule from a mixture of molecules based on specific affinity between molecules e.g., an antigen and antibody binding.

As used herein, the term “matrix” refers to the components of a sample other than the analyte of interest. The matrix can have a considerable effect on the way the analysis is conducted and the quality of the results are obtained.

As used herein, the term “matrix effect” is referred to combined effect of all components of the sample other than the analyte. Components originating from the sample matrix that co-elute with the analytes can interfere with the analyte measurements causing false negative or false positive results.

As used herein, the term “analyte standard” refers to materials containing a known concentration of an analyte.

As used herein, the term “elution solution” refers to a solution that desorbs (or elutes) the analyte from the column material by breaking the specific binding of the analyte with an affinity ligand. Determining the salt, pH and ionic conditions necessary for such functionality of elution solution is well known in the art.

As used herein, the term “rinse solution” refers to a solution that does not desorb or break specific binding of the analyte with an affinity ligand. Rather, the rinse solution is used to carry away disassociated components from the stationary phase of the immunoaffinity column. Typical rinse solutions include aqueous solutions such as water and phosphate buffered saline. Organic solvents, such as methanol, at lower concentration, can also be used.

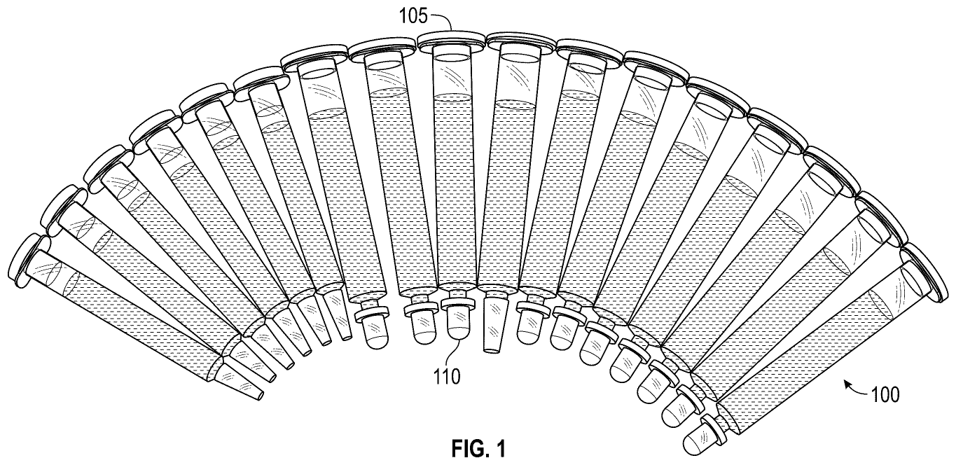

Referring to FIG. 1, the present technology relates to immunoaffinity chromatography columns. FIG. 1 is an image showing numerous immunoaffinity columns 100. Each column 100 includes an inlet 105 where sample is added and an outlet 110 where components exit the column. As shown in FIG. 1, removable caps can be included on both the inlet 105 and exit 110.

Within the immunoaffinity chromatography columns 100 (between inlet 105 and outlet 110) is a stationary phase or support that includes a capture antibody or antigen. When sample is added to the inlet 105 it passes through the column and any analytes that have an affinity to the capture antibody or antigen are absorbed (e.g., bind or associate) to the capture antibody or antigen. The stationary phase or support of immunoaffinity chromatography columns are functionalized to have affinity for a specific analyte. For example, some immunoaffinity columns are functionalized to have affinity for specific mycotoxins. In particular, some immunoaffinity columns are functionalized to have affinity for Ochratoxin A; others may be functionalized to have affinity for Fumonisin; while others have affinity for Aflatoxin. Immunoaffinity chromatography columns can be functionalized with any capture antibody or antigen. That is, immunoaffinity chromatography columns are not limited to affinity for mycotoxins. Mycotoxins are merely illustrative.

Immunoaffinity chromatography columns, also known as IAC columns, are used for sample purification or enrichment. That is, by passing a complex sample through an IAC column functionalized to capture a specific analyte, that analyte can be captured by the column while the other components of the complex sample and other impurities are washed from the column. The analyte within the complex sample absorbs or binds to the capture antibody or antigen within the IAC column. During the washing step, a wash solution that rinses away impurities and other components not absorbed to the capture antibodies or antigens are also released from the column. An elution solution is then used to disassociate the captured analyte from the column. That is, a specific elution solution designed to chemically release the bound analyte is used to elute the analyte from the column and to form an eluate containing both the elution solution and the released analyte.

The problem with conventional methods of using IAC columns resides in the elution solution. In order to release the bound analyte, the elution solution needs to chemically disassociate the analyte from the capture antigen or antibody. To do so, strong acidic solutions, strong basic solutions, strong salt solutions, or strong organic solvents (i.e., the elution solutions) are required to desorb the captured analyte. These elution solutions are retained in the eluate, which is released from the column and make downstream processing challenging. For example, strong salt solutions interfere with mass spectrometry analysis and as a result, an eluate that includes strong salt solution cannot be directly analyzed by a LC-MS analysis.

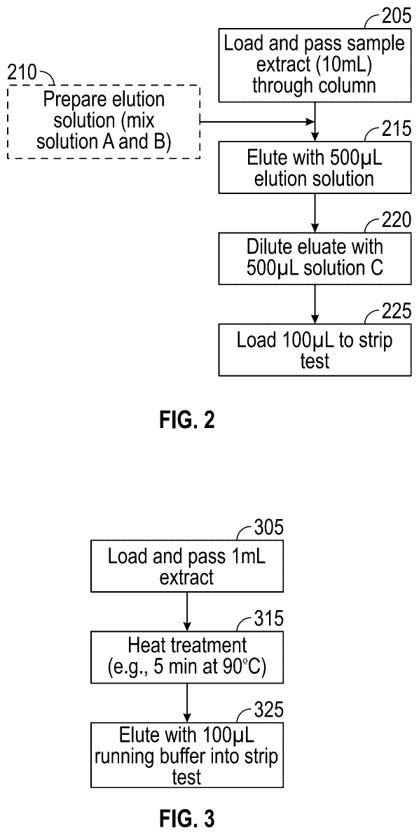

Conventional methods address the problems created by the elution solutions by adding dilution or buffer exchange processing steps after elution from the IAC column to modify the eluate prior to any subsequent analyzation steps. FIG. 2 illustrates the typical method steps needed for eluting and then further processing. Referring to FIG. 2, conventional method 200 includes step 205 in which a sample extract (i.e., a sample solution) is loaded and passed through an IAC column. A wash solution is used to rinse off any impurities during passage of the sample extract through the column. Next an elution solution is used to elute the bound analyte from the IAC column, step 215. As the elution solution is specific to the type of IAC column (i.e., is specific to the chemical disassociation of analyte from the column), the elution solution must be obtained or matched to the column. In certain cases, it is desirable to prepare the elution solution just prior to use (optional step 210). For example and as shown in FIG. 2, an elution solution can be prepared by mixing two or more solutions together just prior to use as an elution solution.

Once the elution solution is used to form an eluate from the column, the eluate must be modified or conditioned for downstream analyzation, such as ELISA testing, HPLC analysis and or mass spectrometry analysis. In step 220 shown in FIG. 2, the eluate is diluted prior to conducting an ELISA test or using a lateral flow strip (step 225). In other conventional methods, buffer exchange processes either in place of the dilution step 220 or in addition to the dilution step 220 may be needed to allow for downstream processing such as HPLC and or LC-MS analysis.

The dilution and buffer exchange process steps (e.g., steps 220) while necessary to address the issues created by the types of elution solutions needed to disassociate the analytes from the columns, are time consuming and introduce possible impurities and matrix effects. In addition to these disadvantages, the dilution and/or buffer exchange process steps add additional volume of liquid to the analysis, which decreases sensitivity and/or requires larger volumes of sample extract to be tested.

The present technology provides a method for elution that does not require a specific elution solution to disassociate the bound analytes from an IAC column. The present technology does not rely on chemical disassociation of the bound analyte and therefore avoids the use of elution solutions. FIG. 3 illustrates a method (300) of the present technology. Method 300 includes loading and passing a sample extract through an IAC column, step 305. Once the sample extract has passed through the column and the analytes therein have absorbed to the IAC column, a heat treatment is applied to disassociate the analytes from the capture antibodies/antigens, step 315. The heat treatment includes heating the IAC column at a temperature which is high enough to denature the analyte (e.g., typically greater than 35 or 40° C.), but is cool enough as to not structurally degrade the IAC column (e.g., typically less than 100° C.). In order to disassociate substantially all of the captured analyte from the column, heat should be applied for a time period between 3 minutes and 60 minutes. More particularly, at lower applied temperatures (e.g., temperatures between 50° C. and 70° C.) longer time periods will be required, e.g., 35 minutes to one hour or more. At higher temperatures, e.g., 75° C. to 95° C., heat may be applied for a shorter time period (e.g., 1 minute (or even 30 seconds) to 25 minutes) to disassociate the analyte from the capture antibody/antigen. One example is to apply heat at 89° C. for 5 minutes. Another example is to apply heat at 90° C. for 2 minutes. A further example is to apply heat at 94° C. for 1 minute.

The final step to elute in method 300 is to rinse the disassociated analyte out of the column with any type of solution. As an eluate is created using a solution of choice (i.e., no need for a conventional elution solution to desorb the analyte), the eluate can be injected or applied directly to a downstream analytical processing technique (step 325) without any intermediate processing or modification steps of the eluate (i.e., no dilution, no buffer exchange). In FIG. 3, the eluate is created by rinsing with a running buffer that is compatible with a lateral strip test for direct processing from the IAC column.

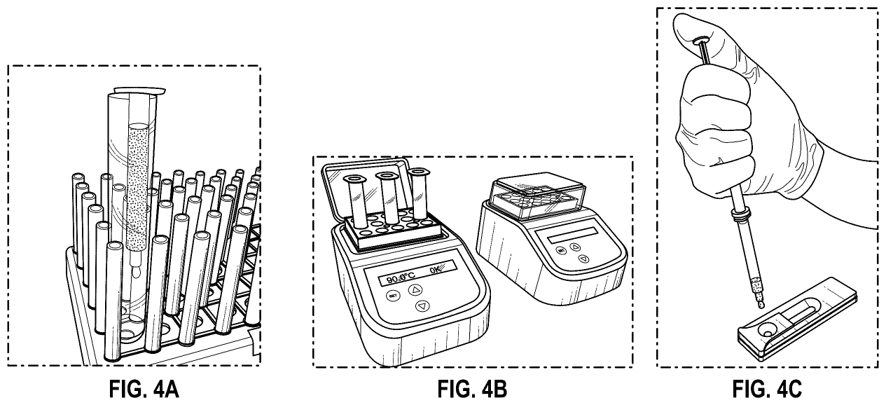

FIGS. 4A, 4B and 4C pictorially illustrate method 300. FIG. 4A illustrates step 305, loading an IAC column with a sample extract. The sample is allowed to flow through the column to allow the analytes within the sample to bind to the capture antibodies/antigens. FIG. 4B illustrates a way to apply a heat treatment to the column, e.g., step 315. As shown in FIG. 4B, the columns can be inserted into a heater having slots designed to receive the columns. In this case, a digital dry bath available from Benchmark Scientific (sold under the tradename MyBlock™ Mini) is used. However, any type of applied energy (e.g., RF radiation, microwave radiation) could be employed to heat the column to temperature for a sufficient time period. After applying the desired heat treatment, any solution is used to rinse the disassociated analyte from the column to form an eluate that can be directly used in a downstream analyzation process, such as, for example injection onto a lateral flow strip as shown in FIG. 4C.

The methods of the present technology provide many advantages over conventional elution methods from IAC columns. For example, the methods of the present technology allow for injection of the eluate into a downstream processing system without modification of the eluate. In some embodiments, the eluate can be injected directly into a HPLC system or a LC-MS system without any modification to the eluate. That is, the solution used to rinse the disassociated analyte from the column can be any solution-and thus can be selected to be compatible with the mobile phase and or a downstream detector of a liquid chromatography system. Further, the eluate need not be diluted in order to be compatible with a lateral flow device or ELISA method or any other downstream detector/detection method. As a result, sensitivity of the downstream analysis is improved as the eluate is not diluted. That is, the detection limit of a downstream analysis is not challenged by the addition of a dilution solution. Further, by limiting the amount of solutions added to the eluant, a smaller amount of sample extract is needed to ensure proper results (i.e., no false negative due to being below detection limits). For example, a comparison of amounts of sample extract and solutions added in method 200 (conventional method using elution solution to disassociate captured analyte) to method 300 (method of the present technology), illustrates this advantage. Method 300 uses 10 times less sample and forms an eluate that contains an enrichment of at least twice that of method 200.

Biochemistry, as well as other fields, can use IAC to selectively purify target compounds from complex samples. Some compounds that have been isolated by this process include proteins, glyocproteins, carbohydrates, lipids, bacteria, viral particles, drugs and environmental agents. Further, IAC columns have been used in combination with a detector to measure or identify an amount of a captured analyte from a complex sample. Some examples of analytes that have been measured by this approach include human serum albumin, recombinant tissue-type plasminogen activator, recombinant antithrombin III, IgG, Escherichia coli, isoproturon, phenylurea herbicides, benzidine, dichlorobenzidine, aminoazobenzene, azo dyes, triazine, diethylstilbestrol, acetylcholinesterase, transferrin, and insulin. The methods of the present technology can be used to detect any of these analytes.

Sample extracts can be made from urine, phlegm, food, crop, water and soil extracts. Examples of analytes that have been examined by this approach include α₁-anti-trypsin, atrazine, benzylpenicilloyl-peptides, bovine serum albumin, carbendazim, chloramphenicol, cortisol, clenbuterol and phenytoin, among others. Additional analytes include toxins, such as mycotoxins (e.g., aflatoxin, fumonisin, zearalenone, ochratoxin, DON, T2/HT2). For solid samples, such as, for example, food, crops and soil, the solids can be crushed or obtained in a powdered form. Water or organic solvents are added to the powdered sample and, in most cases, are filtered using filter paper and possibly a glass-fiber filter, to form a sample solution (i.e., a sample extract) for loading and passing through the IAC column.

In one or more embodiments, the analyte to be captured by the IAC column is selected from toxins, allergens, or antibiotics. In one embodiment, the toxins are mycotoxins and are selected from the group of aflatoxin, ochratoxin, deoxynivalenol, nivalenol, T2/HT2 toxin, patulin, zearalenone, citrinin, fumonisin or their analogs. In another embodiment, the aflatoxin is selected from the group of aflatoxin B1, aflatoxin B2, aflatoxin G1, aflatoxin G2, aflatoxin M1, or aflatoxin M2.

In some embodiments, the IAC column is conditioned before use with a saline solution, such as, for example, phosphate buffered saline. However, conditioning is not necessary and in some instances is not desired.

In the present technology any type of solution can be used to rinse the disassociated analyte from the column after application of the heat treatment. In general, the rinse solution can be selected based on its compatibility with downstream analytic processing apparatus or steps. For example, aqueous solutions, such as water or saline can be used. In other embodiments, organic solvents such as methanol or a combination of water and methanol can be used. In some embodiments the rinse solution can be the same solution used for forming the sample solution. For example, if a sample is formed from a mixture of a powdered food source and water and methanol blend, the rinse solution can also be the same water and methanol blend used in the formation of the sample extract.

The methods of the present technology can be used in connection with diagnostic or analytical detection kits. Kits in accordance with the present technology include an IAC column, a consumable analytical processing device (e.g., a lateral flow strip, a LC column) and a vial of a rinse solution compatible with the consumable analytical processing device. The kit also includes instructions for performing a heat treatment step on the IAC column after a sample solution has been loaded and passed through the IAC column.

An illustrative example of such a kit, is a kit directed to testing for ochratoxin in a food source such as cocoa powder. The kit includes an IAC column having a stationary phase or support functionalized to capture ochratoxin (e.g., OchraTest IAC columns, available from Vicam, Milford, MA), a lateral flow strip test that detects the presence of ochratoxin (e.g., Ochra-V strips, available from Vicam), and a vial of a compatible rinse solution, such as methanol, water, or combinations thereof). The kits also include instructions for performing the method, and in some instances can include one or more of pipette tips, filter paper, sample extraction solution, a sample reader, such as a Vertu™ TOUCH reader available from Vicam, Milford, MA); and or calibration information or calibration samples used in connection with the sample reader.

The following examples illustrate the above methods and kits of the present technology.

### EXAMPLES

The following examples are included to demonstrate preferred embodiments of the invention. It should be appreciated by those of skill in the art that the techniques disclosed in the examples which follow represent techniques discovered by the inventor to function well in the practice of the technology, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the technology.

Example 1: Testing of elution temperature and time period for Aflatoxin recovery

To study temperature effect on aflatoxin B1 elution, seven aflatoxin samples were created using aflatoxin B1 analyte standard available from Sigma Aldrich, part number CRM 44647. Specifically, 5 nanograms of aflatoxin B1 standard was spiked into a 10 ml 1xPBS solution. This solution was run through an IAC column functionalized for the capture of aflatoxin (Aflatest IAC column, available from Vicam, lot P2309DR).

The seven aflatoxin samples were loaded and run through the Aflatest IAC columns at a flow rate of about 1 drop every 2 seconds. The columns were washed with 2 ml of purified water (Mini-Q purification system from Millipore) and blown dry. One column was then subjected to conventional elution techniques and the remaining six columns were subjected to various heat treatments. For the column subjected to conventional elution techniques, 1 ml of methanol was used for elution to create an eluate. The eluate was then diluted with 1 ml of purified water. Then 10 μL of the diluted eluate was injected into a UHPLC column for analysis.

The remaining six columns were subjected to the heat treatments for either 5 minutes or 10 minutes using a heat block set to 60° C., 70° C. or 90° C. That is, a first column after loading and passing of the sample was heated using a heat block set to 60° C. for 5 minutes; a second column after loading and passing the sample was heated using a heat block set to 60° C. for 10 minutes; a third column after loading and passing of the sample was heated using a heat block set to 70° C. for 5 minutes; a fourth column after loading and passing the sample was heated using a heat block set to 70° C. for 10 minutes; a fifth column after loading and passing of the sample was heated using a heat block set to 90° C. for 5 minutes; and a sixth column after loading and passing of the sample was heated using a heat block set to 90° C. for 10 minutes. After the specific heat treatment was applied the columns were rinsed with 1 mL of purified water to form an eluate. To this eluate 1 mL of methanol was added and mixed prior to injection of 10 μL into a UHPLC column for analysis.

Table 1 below provides the amount of aflatoxin recovered together with its percentage of recovery for the seven samples. The results presented in Table 1 below show that a heat treatment of 90° C. for 10 minutes disrupts aflatoxin binding to capture antibodies as a recovery of over 85% of the standard injected into the IAC column was obtained. The amount recovered for this particular heat treatment (90° C., 10 minutes) is equivalent to the recovery of sample in which an organic solvent (methanol) was used for elution/chemical disassociation of the aflatoxin from the capture antibodies in the IAC column.

TABLE 1
Aflatoxin B1 recovered from IAC Column

| | | | | Elution |
|---|---|---|---|---|
| | Applied Heat Treatment - Elution Technique | | | Solution |
| | 60° C. | 70° C. | 90° C. | Methanol |
| 5 minutes | 0.225 (4.5%) | 0.34 (6.8%) | 1.935 (38.7%) | |
| 10 minutes | 0.335 (6.7%) | 0.6 (12%) | 4.26 (85.2%) | |
| | | | | 4.23 (84.6%) |

Example 2: Evaluation of Sensitivity of Lateral Flow Test Results using Different Elution Techniques.

To illustrate the advantageous of the present technology the following example is provided comparing a test/control response read from a lateral flow test after applying various elution techniques. Specifically, cocoa powder samples were spiked with a known amount of ochratoxin (e.g., less than 0.5 ppb; 2.1 ppb; or 4.3 ppb). Sample solutions were made from the cocoa powder spiked with ochratoxin by measuring 2.5 grams of the spiked sample, adding 10 mL of 80% methanol in water and mixing for 2 minutes. 40 mL of 1 xPBS was added and mixed to the sample solutions and the extract was filtered through paper (P8 filter paper from Fisherbrand, part number 09-795D). The filtered sample solutions (10 mL) were then applied (i.e., 1 drop per sec) to an IAC column functionalized for affinity to ochratoxin (OchraTest IAC columns, available from Vicam); one of the following three elution methods were applied and the eluates were tested using a lateral flow strip (Ochra-V strip, available from Vicam). It is noted that for elution methods A and B, the eluates were conditions (i.e., diluted) prior to the lateral flow test; for elution method C, which used a heat treatment no dilution was applied to the eluate.

For elution method A, captured analyte was eluted using an elution solution (elution solution A) containing 0.1 M glycine, 0.1 M HC1 at a pH of 1.8; to the eluate 500 μL of a solution containing a phosphate salts, saline, and surfactant (Aqua Premix solution available from Vicam) and 100 μL of 0.5 NaOH solution was added to condition the eluate prior to injection on a lateral flow test strip designed to detect ochratoxin (Ochra-V strip, available from Vicam).

For elution method B, captured analyte was eluted using a different elution solution (elution solution B) containing 0.1 M glycine, 0.108 M HC1, 2% Tween-20 (a non-ionic surfactant including polysorbate available from Thermo Fisher Scientific Inc.) and 1% sodium dodecyl sulfate (a detergent). To the eluate, 500 μL of 0.1 M NaOH, 2.48% NaCl, and 0.56 Na₂HPO₆ was added to condition the eluate prior to injection on the lateral flow strip test (Ochra-V strip, available from Vicam).

For elution method C, capture analyte was released from the IAC column by incubating the column in a water bath set at 90° C. for 10 minutes. The IAC column was rinsed with a saline solution including a surfactant (a running buffer known as Aqua Premix solution available from Vicam) to form the eluate. The eluate was then directly injected onto the lateral flow strip (Ochra-V Strip, available from Vicam)

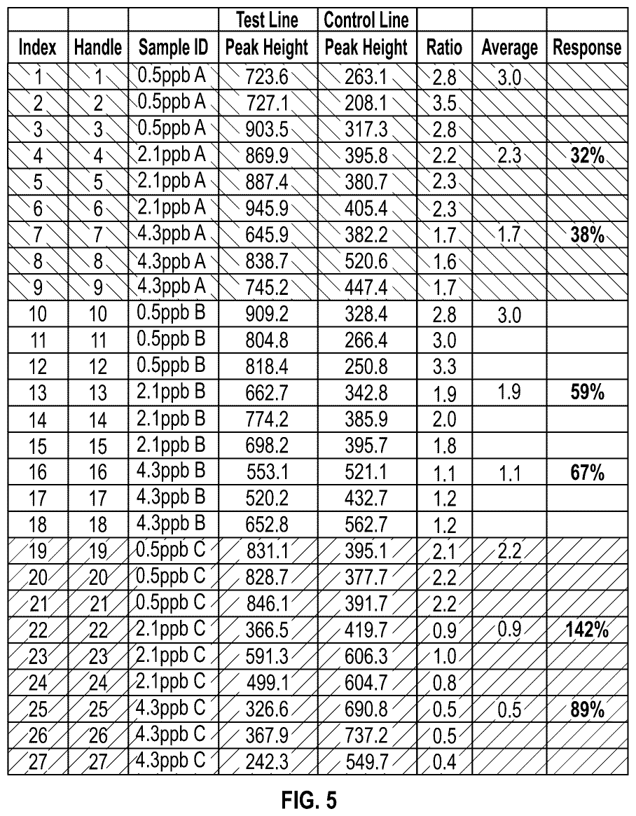

After injection on the lateral flow strips, the tests were incubated for 5 minutes and read with a Vertu™ lateral flow strip reader (available from Vicam). FIG. 5 presents three test results per spiked concentration level for each of the three different elution methods. Samples ID nomenclature in FIG. 5 identifies the concentration of the ochratoxin spiked into the sample (i.e., 0.5 ppb, 2.1 ppb, or 4.3 ppb) as well as the elution method used (i.e., A, B, or C). As can be seen viewing the far-right column of the table illustrated in FIG. 5, elution method C (i.e., the method of the present technology) provided the highest response (142% and 89%) versus the conventional methods which required dilution prior to injection onto a lateral flow strip.

Further modifications and alternative embodiments of various aspects of the technology will be apparent to those skilled in the art in view of this description. Accordingly, this description is to be construed as illustrative only and is for the purpose of teaching those skilled in the art the general manner of carrying out the technology. It is to be understood that the forms of the technology shown and described herein are to be taken as examples of embodiments. Elements and materials may be substituted for those illustrated and described herein, parts and processes may be reversed, and certain features of the invention may be utilized independently, all as would be apparent to one skilled in the art after having the benefit of this description of the technology.

Changes may be made in the elements described herein without departing from the spirit and scope of the technology as described in the following claims.

What is claimed is:

1. A method of eluting a bound analyte from an immunoaffinity column, the method comprising:
passing a sample solution comprising an analyte of interest through the immunoaffinity column to associate a portion of the analyte of interest to stationary phase material of the immunoaffinity column;
heating the immunoaffinity column at a temperature between about 65° C. to about 95° C. for a time period between about 2 minutes and about 30 minutes to disassociate the portion of the analyte of interest from the stationary phase material; and
rinsing the immunoaffinity column with a solution to create an eluate including the disassociated portion of the analyte of interest.

2. The method of claim 1, further comprising injecting the eluate into a downstream processing system without modification of the eluate.

3. The method of claim 1, wherein the solution for rinsing the immunoaffinity column is an aqueous solution.

4. The method of claim 1, wherein prior to passing the sample solution through the immunoaffinity column, the immunoaffinity column is conditioned using phosphate buffered saline solution.

5. The method of claim 1, wherein the column is heated at a temperature of about 90° C.

6. The method of claim 5, wherein the column is held at temperature for about 10 minutes.

7. The method of claim 5, wherein the column is held at temperature for about 7 minutes.

8. The method of claim 5, wherein the column is held at temperature for about 5 minutes.

9. The method of claim 2, wherein the eluate is injected into a HPLC column.

10. The method of claim 2, wherein the eluate is injected on to a lateral flow strip.

11. The method of claim 10, wherein a reader is used to determine a ratio of peak height of a test line to peak height of a control line.

12. The method of claim 2, wherein the eluate is used in an ELISA test.

13. The method of claim 2, wherein the eluate is injected into a LC-MS system.

14. The method of claim 1, wherein the sample solution includes a powdered food sample mixed with water.

15. The method of claim 1, wherein the sample solution includes a powdered food sample mixed with an organic solvent.

16. The method of claim 14, wherein the sample solution is filtered prior to passing the sample solution through the immunoaffinity column.

17. The method of claim 15, wherein the sample solution is filtered prior to passing the sample solution through the immunoaffinity column.

18. The method of claim 1, wherein the immunoaffinity column is a mycotoxin immunoaffinity column.

19. The method of claim 18, wherein the mycotoxin immunoaffinity column is an Ochratoxin immunoaffinity column.

20. A kit for eluting a captured analyte from an immunoaffinity column, the kit comprising:
an immunoaffinity column;
a consumable analytical processing device having an inlet for injection of the eluate of claim 1; and
a vial of a rinse solution compatible with the consumable analytical processing device.
